(19) United States
(12) Patent Application Publication
(10) Pub. No.: US 2026/0325878 A1
(43) Pub. Date: Oct. 22, 2026

(54) PHOTON BEAM BOLUS DESIGN METHOD FOR PHOTON BEAM RADIATION THERAPY

(71) Applicant: SWEAT HOLDINGS, LLC, Sanford, FL (US)

(72) Inventor: Benjamin E. Nelms, Merrimac, WI (US)

(21) Appl. No.: 19/643,674

(22) Filed: Apr. 9, 2026

Related U.S. Application Data
(60) Provisional application No. 63/789,587, filed on Apr. 16, 2025.

(51) Int. Cl. A61N 5/10

(52) U.S. Cl. CPC A61N 5/1031; A61N 5/1039; A61N 5/1077; A61N 2005/1085

(57) ABSTRACT

A method for designing a photon beam bolus for use in photon beam radiation therapy includes receiving three-dimensional imaging data of a patient and delineations of anatomic regions including an external patient contour and an internal target volume. A bolus design algorithm is used to generate a bolus geometry that includes an inner surface conforming to the external patient contour, and an outer surface determined based on the internal target volume and megavoltage photon beam dose deposition characteristics to be applied to the internal target volume. A profile of the photon beam bolus defines a spatially-varying thickness between the inner surface and the outer surface.

### CROSS-REFERENCE TO RELATED APPLICATIONS

The present application claims the benefit of U.S. Provisional Patent Application Serial No. 63/789,587 filed Apr. 16, 2025, all of which is fully incorporated by reference.

### TECHNICAL FIELD

The present disclosure relates to radiation therapy, and, more particularly, to a radiation bolus used in external photon beam radiation therapy.

### BACKGROUND

External photon beam radiation therapy uses photon beams to deliver radiation doses in the treatment of cancer. Megavoltage (MV) photon beams are used to deliver energy (i.e., deposit radiation doses) inside of humans, with the aim to kill cancer cells and remove (or shrink) tumors.

The physics of radiation deposition from megavoltage photon beams results in a radiation “buildup region” where the first, i.e., shallowest, depths of tissue encountered, despite receiving the highest strength of beam, receive a lower absorbed dose relative to deeper depths. Therefore, the dose deposited by the beam will first increase with depth before it eventually starts becoming lower as the beam attenuates deeper in the patient. The buildup region for therapeutic MV beams is typically on the order of 10-20 mm.

Some radiation target volumes (e.g., cancerous regions) are partially, or sometimes fully, at shallow depths inside the patient’s body. If the shallow depths are treated with direct MV radiation beams, they are treated inadequately, or at least inefficiently, because the shallow depths overlap the low-dose buildup region. This is not ideal.

To treat the shallow target volumes to higher doses, i.e., beyond the lower-dose buildup region, clinicians may place a photon beam bolus on the patient surface. The bolus is a material with a composition and density close enough to human soft tissue that essentially expands the patient’s external surface, in turn making the target volume deeper. The radiation buildup then occurs in the region of the bolus, and the radiation dose deposited is higher by the time it enters the target volume than it would have been without the bolus.

Photon beam boluses have historically been uniform-thickness slabs of flexible material that are placed on the patient surface. The thickness is chosen based on the beam energy used to provide enough buildup. These generic boluses are not custom-fit, and they often do not fit well to patient exterior shapes, especially in complex regions (e.g., scalp, nose, ears, head/neck, arms, pelvis, arms, and legs).

More recently, instead of generic slabs of flat material, uniform thickness boluses(UTB) can be manufactured to fit the patient by transferring the bolus shape specifications to a manufacturing system for fabrication using a milling machine (e.g., cut out of machinable wax), using a 3D printer, or by manufacturing a 3D mold first, then curing a flexible material (e.g., silicone) inside the mold. These manufactured boluses are patient-specific and custom fit, to replace the generic slabs, but they are still uniform thickness.

Uniform thickness boluses are extensions of the patient, and therefore, for better or worse, they preserve the shape of the patient. Depending on the body site, these surfaces can be complex (ears, eyes, face, anterior pelvis, etc.) and with no consideration of the underlying target volume. This creates many challenges.

### SUMMARY

A method for designing a photon beam bolus for use in photon beam radiation therapy includes receiving three-dimensional imaging data of a patient and delineations of anatomic regions including an external patient contour and an internal target volume. A bolus design algorithm is used to generate a bolus geometry that includes an inner surface conforming to the external patient contour, and an outer surface determined based on the internal target volume and megavoltage photon beam dose deposition characteristics to be applied to the internal target volume. A profile of the photon beam bolus defines a spatially-varying thickness between the inner surface and the outer surface.

The bolus design algorithm may be configured to generate the bolus geometry by creating an external shape to influence the megavoltage photon beam dose deposition characteristics in the target volume within the created external shape, and subtracting the external patient contour from the created external shape.

The bolus geometry may be configured for any photon beam direction.

The bolus design algorithm may be configured to add alignment guides to the outer surface of the photon beam bolus, with the alignment guides being used to accurately position the photon beam bolus on the patient. The alignment guides may be configured as grooves or ridges.

The external patient contour may include a sensitive anatomical region that a patient might prefer to remain private, e.g., the shape of external male or female genitalia. The bolus design algorithm may be configured to shape the outer surface of the bolus geometry to anonymize the sensitive anatomical region.

The bolus design algorithm may be configured to apply a user-defined margin to the bolus geometry. The outer edges of the user-defined margin may be tapered and smooth.

The bolus design algorithm may be configured to shape the inner surface of the bolus geometry to correct small-scale imperfections in the external patient contour, such that the photon beam bolus more accurately conforms thereto.

In response to the internal target volume corresponding to noncontiguous anatomical regions, the bolus design algorithm may be configured to generate a plurality of separate bolus geometries. Each bolus geometry may correspond to a respective region, and is configured for independent manufacture. A user may be enabled to select one or more of the plurality of bolus geometries for use.

The bolus geometry may be exported for manufacture of the photon beam bolus, or to a treatment planning system.

Another aspect is directed to a computing device that includes a memory and processor. The memory receives three-dimensional imaging data of a patient and delineations of anatomic regions including an external patient contour and an internal target volume. The processor is coupled to the memory and is configured to execute a bolus design algorithm for designing a photon beam bolus for use in photon beam radiation therapy. The bolus design algorithm may be configured to generate a bolus geometry that includes an inner surface conforming to the external patient contour, and an outer surface determined based on the internal target volume and megavoltage photon beam dose deposition characteristics to be applied to the internal target volume. A profile of the photon beam bolus may define a spatially-varying thickness between the inner surface and the outer surface.

Yet another aspect is directed to a photon beam bolus for use in photon beam radiation therapy. The photon beam bolus includes a body comprising a bolus geometry that includes an inner surface configured to conform to an external patient contour, and an outer surface defined based on an internal target volume and megavoltage photon beam dose deposition characteristics associated with the internal target volume. The bolus geometry may define a spatially-varying thickness between the inner surface and the outer surface.

Yet a further aspect is directed to a non-transitory computer readable medium having computer-executable instructions for causing a computing device to execute a bolus design algorithm as described above.

### BRIEF DESCRIPTION OF THE DRAWINGS

FIG. 1 is a block diagram of a computing device configured to execute a bolus design algorithm in which various aspects of the disclosure may be implemented.

FIG. 2A is a CT image of a patient’s head with a uniform thickness photon beam bolus placed over an internal target volume according to the prior art.

FIG. 2B is a CT image of the patient’s head illustrated in FIG. 2A but with an optimized, spatially-varying thickness bolus placed over the internal target volume.

FIG. 3 is a high-level workflow diagram for a treatment planning facility using the bolus design algorithm illustrated in FIG. 1.

FIG. 4 is a detailed summary workflow diagram of the bolus design algorithm illustrated in FIG. 1.

FIG. 5 is a screen shot of user-entered design parameters for the bolus design algorithm illustrated in FIG. 1.

FIG. 6A is a CT image of a male’s anterior pelvis with an optimized thickness bolus placed over a sensitive anatomical region to be treated.

FIG. 6B is a 3D view of the patient illustrated in FIG. 6A with the optimized, spatially-varying thickness bolus protecting the privacy of male genitalia for the patient.

FIG. 7A is a 3D view of a patient’s head with an optimized thickness bolus placed thereon that includes alignment guides and a conformal outer margin.

FIG. 7B is 3D views of the optimized thickness bolus illustrated in FIG. 7A, one with concave alignment guides and one with convex alignment guides.

FIG. 8 is a flowchart for designing the photon beam bolus illustrated in FIG. 1.

### DETAILED DESCRIPTION

The present description is made with reference to the accompanying drawings, in which exemplary embodiments are shown. However, many different embodiments may be used, and thus the description should not be construed as limited to the particular embodiments set forth herein. Rather, these embodiments are provided so that this disclosure will be thorough and complete. Like numbers refer to like elements throughout.

A key limitation of conventional photon beam boluses lies in their uniform thickness design. One challenge of a uniform thickness bolus (UTB) is in terms of manufacturing. Modeling a bolus strictly off the external contour of the patient can lead to complex and imperfect bolus designs. This is especially true if the model of the patient surface, as generated by contouring of the planning CT (or MR) images, is imperfect or pixelated.

Another challenge is directed to dosimetric planning. The complex shape (even with the bolus) can limit the achievable plan quality in terms of coverage of targets and sparing of healthy tissue compared to a more regularly-shaped patient volume. Even if the patient shape is not complex, a uniform thickness bolus is an approximate of how much build up to add, and it is not designed based on the underlying target itself.

Moreover, a complex and/or non-customized bolus can cause inverse-planned beam optimization to have to “work harder” to produce the desired target coverage while sparing organs-at-risk inside the patient. This often increases beam complexity and/or planning time. Complex exterior volumes and/or inadequate uniform thickness boluses can strain the dose calculation accuracy inside the patient volume that contains important targets and organs-at-risk.

Yet another challenge is that uniform thickness boluses may be hard to position and align accurately on the patient’s skin with respect to how they were simulated in planning. There is also a challenge with privacy and human comfort. Boluses that mimic the patient shape will, in certain anatomical sites, mimic what would commonly be considered private anatomy, e.g., bolus designed for the anterior pelvis regions of both male and female patients. The radiation therapist may have to position that bolus every day, and that patient may feel exposed every day, and self-conscious or dehumanized.

As used herein, the term “internal target volume” represents any or all treatment volumes inside the patient body (tumors, gross/clinical/planning target volumes, etc.) where a prescriptive dose is to be deposited.

As used herein, the term “external patient contour” represents a mapped 3D surface of the exterior of the patient, e.g., as defined by a multitude of axial contours drawn on a series of medical images.

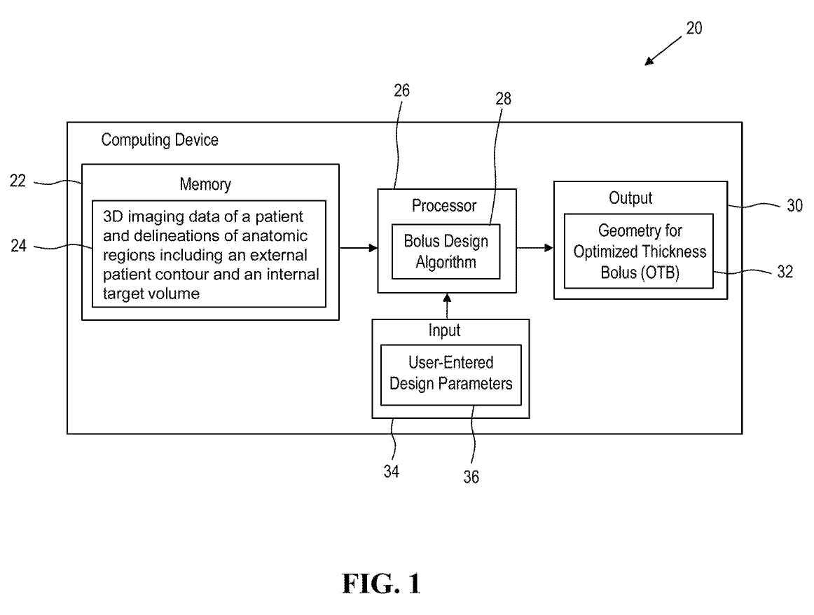

Referring initially to FIG. 1, the above challenges associated with a uniform thickness bolus are addressed by a bolus design algorithm 28 to be executed by a computing device 20. As will be discussed in greater detail below, the bolus design algorithm 28 is configured to generate geometry 32 for a photon beam bolus that has a profile defining a spatially-varying thickness between inner and outer surfaces of the photon beam bolus. The generated geometry 32 for the photon beam bolus is advantageously based on the internal target volume within the patient and not as an extension of the external patient contour surface expanded by a uniform thickness. The photon beam bolus will also be referred to as an optimized thickness bolus or OTB.

The computing device 20 includes a memory 22 to receive three-dimensional (3D) imaging data of a patient and delineations of anatomic regions 24 including an external patient contour and an internal target volume.

The bolus design algorithm 28 will also require a host of design parameters. Some will be entered by the user, some will be derived from the user-defined data, others will be algorithm parameters modeling the physics of external beam dose deposition of photon beams, and others will be hard-coded. An input 34 is for the user to enter the user-defined design parameters 36.

A processor 26 is coupled to the memory 22 and to the input 34 and is configured to execute the bolus design algorithm 28 to generate the bolus geometry 32. The bolus geometry 32 is provided to an output 30. The bolus geometry 32 may then be exported for manufacture of the optimized thickness bolus, or to a treatment planning system.

The bolus geometry 32 includes an inner surface conforming to the external patient contour, and an outer surface determined based on the internal target volume and megavoltage photon beam dose deposition characteristics to be applied to the internal target volume.

The optimized thickness bolus simplifies the external shape of the irradiated volume, so that the beams can be optimized for the internal target volumes without the added optimization challenges caused by insufficient buildup material (portions of shall target) and/or a complex external shape. The optimized thickness bolus can help simplify the complexity of beams, which may advantageously lower treatment time and require less radiation exposure and energy used. Less complex beams will also have a lower risk of systematic and random errors.

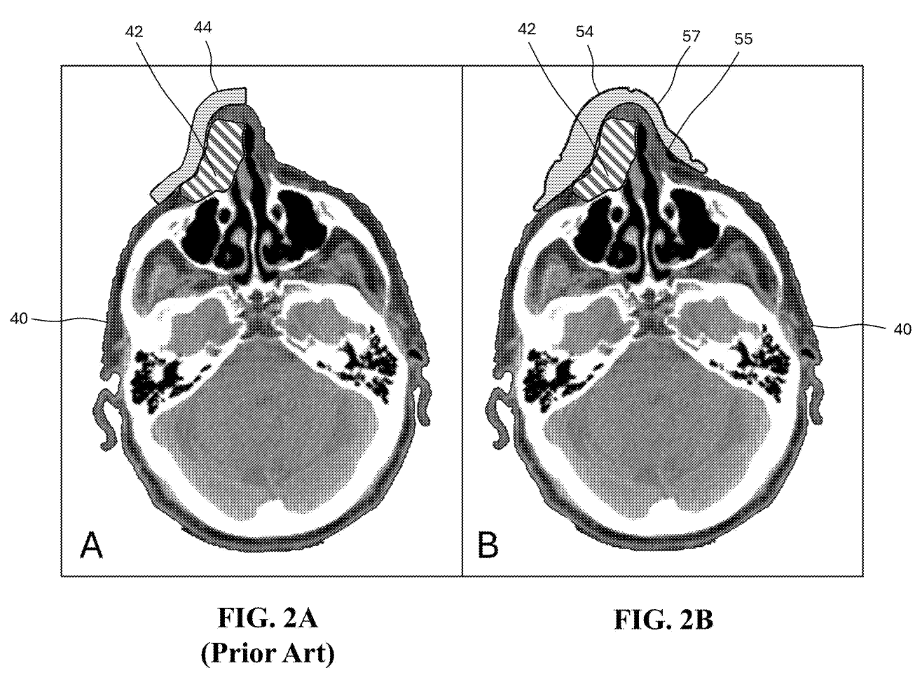

A CT image 40 of a patient’s head is illustrated in FIGS. 2A-2B. The internal target volume 42 to be treated is in the nasal area that is partially shallow and not symmetric with respect to the patient’s midline. As illustrated in FIG. 2A, the internal target volume 42 would not receive enough buildup in some areas (and perhaps over the whole area) if a conventional uniform thickness bolus 44 of 5 or 10 mm is used.

However, as illustrated in FIG. 2B, an optimized thickness bolus 54 with goal buildup thickness selected based on beam energy (e.g., 15-16 mm for a 6 MV photon beam) ensures adequate buildup in 3D across all possible beam entry angles, even for rotational (i.e., arc) beams. This will allow the dose optimization engine in the treatment planning system to better achieve the desired target coverage, even at the shallowest target depths.

The optimized thickness bolus 54 includes an inner surface 55 conforming to the external patient contour, and an outer surface 57 determined based on the internal target volume and megavoltage photon beam dose deposition characteristics to be applied to the internal target volume. As noted above, a profile of the optimized thickness bolus 54 defines a spatially-varying thickness between inner and outer surfaces of the photon beam bolus.

As will be discussed in greater detail below, the optimized thickness bolus 54 may also be configured to include alignment guides 170. Alignment guides 170 are used for visual alignment using treatment machine systems and/or room alignment systems, e.g., laser alignment systems.

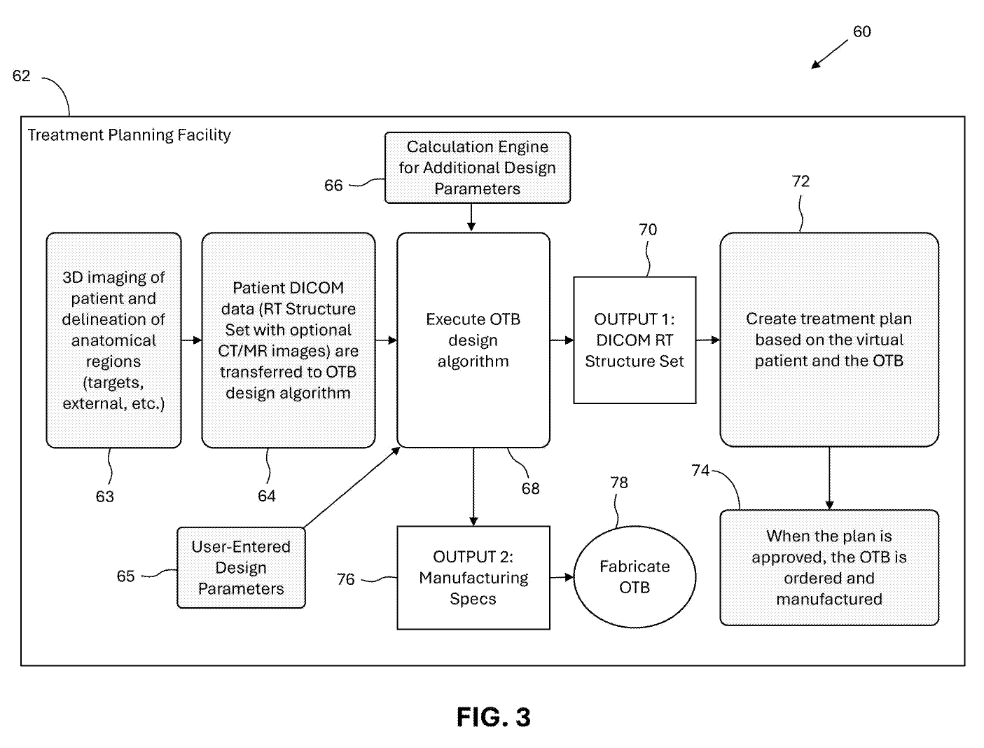

A high-level workflow diagram 60 illustrated in FIG. 3 for a treatment planning facility 62 using the bolus design algorithm 28 will be discussed. The treatment planning facility 62 is to generate a radiation treatment plan based on patient anatomical data and prescribed dose parameters.

At Block 63, three-dimensional imaging data of a patient and delineations of anatomic regions including an external patient contour and an internal target volume are received. A goal buildup bolus thickness is also received. The goal buildup thickness of the optimized thickness bolus 54 may be 15 mm, for example.

The optimized thickness bolus 54 design will ensure this goal thickness of material (combination of bolus material and patient tissue) will be encountered for any possible beam geometry. This provides the desired building up of dose prior to the internal target volume while simplifying the external irradiated shape. This custom shape is created via the bolus design algorithm 28.

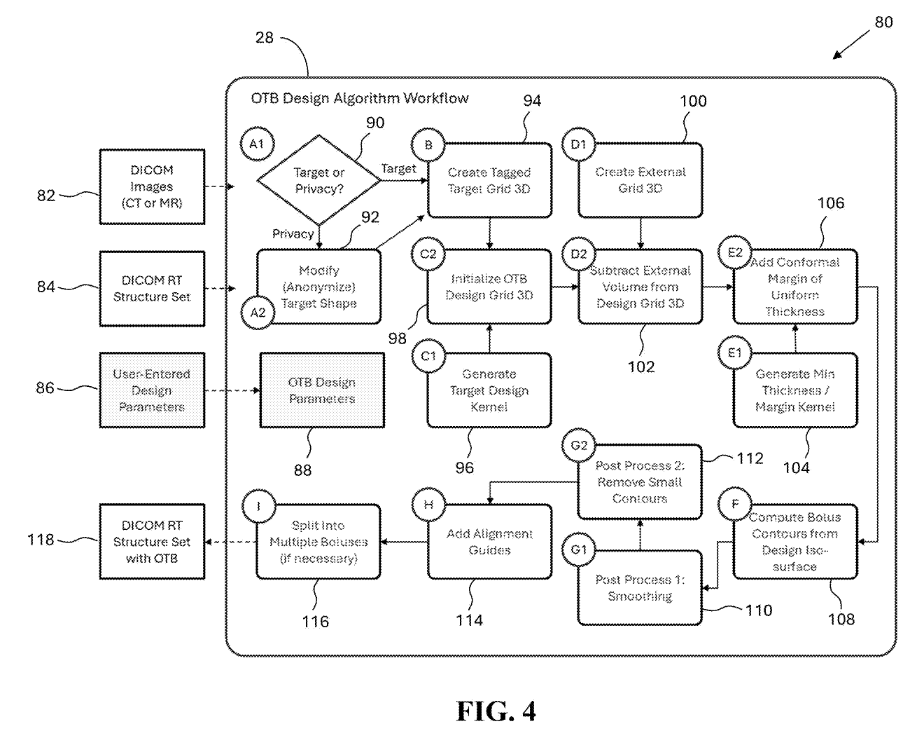

Patient DICOM data (DICOM RT Structure Set with optional DICOM CT/MR images) are transferred to the bolus design algorithm 28 at Block 64. DICOM is a standard for digital imaging and communications in medicine, and is widely implemented in radiology as the standard for diagnostic imaging. The bolus design algorithm 28 is specifically designed to be used by technology found in the treatment planning facility 62. A detailed workflow of the bolus design algorithm 28 will be discussed below in reference to FIG. 4.

The user-defined parameters 36 are transferred to the bolus design algorithm 28 at Block 65. The user-defined parameters 36 include identification of the target region of interest (ROI), the external patient ROI, the primary purpose (target buildup/dosimetry or patient privacy and surface simplification), the goal buildup distance (usually based on the energy of the treatment beams), the outer margin size, the bolus material (e.g., silicone), the alignment guide design (e.g., concave grooves or convex ridges), and the orthogonal plane locations at which to add the alignment guides on the outer (visible) surface of the bolus.

Addition design parameters needed by the bolus design algorithm 28 are provided by a calculation engine at Block 66. The calculation engine may be separate from the bolus design algorithm 28, as shown, or may be integrated within the bolus design algorithm 28. The calculation engine typically generates additional parameters that the user does not directly input but are essential for accurate dose buildup and shaping. Parameters may include derived geometric, radiological, and dosimetric parameters, for example.

The bolus design algorithm 28 is executed at Block 68. Execution of the bolus design algorithm 28 may be performed locally by the treatment planning facility 62, or alternatively, may be outsourced to a remote computing system 20. The remote computing system 20 may be a cloud-based processing service or an external treatment planning provider. Upon execution of the bolus design algorithm 28, a customized, non-uniform thickness photon beam bolus (i.e., an optimized, spatially-varying thickness bolus) 54 is generated for the patient.

Design specs for the 3D optimized thickness bolus 54 are output at Block 70 in a DICOM RT Structure Set format from the bolus design algorithm 28. This corresponds to output 1. A treatment plan is created at Block 72 based on the virtual patient and the optimized thickness bolus 54.

The treatment plan for the patient is optimized for the patient with the optimized thickness bolus 54. Upon approval of the treatment plan, the optimized thickness bolus 54 is ordered and manufactured at Block 74. The order may sent in the DICOM RT Structure Set format to a third party to process the DICOM inputs, and manufacture the non-uniform thickness photon beam bolus 54.

As an alternative, output 2 76 from the bolus design algorithm 28 is in the form of manufacturing specs. This allows an order to be placed for manufacture of the optimized thickness bolus 54. The order may be received by a third party at Block 78 to manufacture the non-uniform thickness photon beam bolus 54. The manufacture may be performed in-house within the treatment planning facility 62 if the facility has a 3D printing and/or high-end machine shop. Alternatively, the manufacture may be outsourced.

Referring now to FIG. 4, a detailed summary workflow diagram 80 of the bolus design algorithm 28 will be discussed below. The workflow is labeled both as Steps A-I and Blocks 90-116, with an emphasis on the block labeling. A number of inputs are required by the bolus design algorithm 28.

One input is DICOM images to be provided at Block 82. Digital Imaging and Communications in Medicine (DICOM) image data comprises a standardized format for storing, transmitting, and managing medical imaging information. In radiation therapy planning, DICOM image sets typically include volumetric datasets such as CT, MR, and PET images acquired as sequential axial slices. These slices collectively define a three-dimensional patient-specific anatomical volume in a consistent spatial coordinate system. Each image contains both pixel data and associated metadata, including slice thickness, pixel spacing, patient orientation, and acquisition parameters. This enables accurate spatial reconstruction and registration across modalities. This standardized structure facilitates interoperability between imaging systems and treatment planning systems, and provides the foundational dataset for contouring, segmentation, and dose calculation workflows.

Another input is a DICOM RT Structure Set to be provided at Block 84. The DICOM RT Structure Set is a radiotherapy-specific DICOM file that stores delineated anatomical structures as a set of region-of-interest (ROI) contours referenced to the underlying imaging dataset. Included are user-defined or automatically generated contours representing targets (e.g., tumors) and organs-at-risk, typically defined on individual axial slices that together can be rendered to represent volumes across the entire range of axial slices. Each structure is encoded with spatial coordinates in the same frame of reference as the DICOM image series, ensuring precise geometric alignment for treatment planning. The RT Structure Set also includes metadata such as structure names, types, and identifiers, enabling downstream use in dose optimization, treatment evaluation, and quality assurance within radiation therapy planning systems. The calculation can proceed with only the DICOM RT Structure Set, but that would not enable the patient tissue density corrections to be factored into the bolus design algorithm 28.

The bolus design algorithm 28 will require a host of design parameters. Some will be entered by the user at Block 86, and some will be derived from the user-defined data at Block 88. Other design parameters will be algorithm parameters modeling the physics of external beam dose deposition of photon beams, and others will be hard-coded.

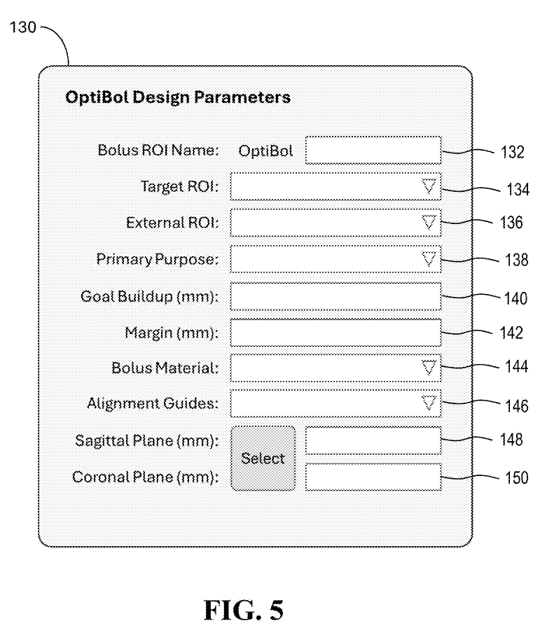

The user-entered design parameters are to be provided at Block 86 which are received by Block 88 within the bolus design algorithm 28. A screenshot 130 of some of the design parameters to be supplied by the user is illustrated in FIG. 5.

A user-defined name of the region of interest (ROI) of the resulting optimized bolus is entered in input field 132. Input field 134 is for a user-defined target ROI that is as already defined as a set axial contours. This defines the internal volume to be treated to prescribed dose, which may be a single target ROI or a union of multiple target ROIs. Input field 136 is a user-defined external ROI that defines the external patient surface.

Input field 138 is for the user to identify a primary purpose of the bolus. The primary purpose may be for target buildup and dosimetry or for patient privacy and surface simplification. If an external patient contour includes a sensitive anatomical region, then patient privacy is selected. Here, the bolus design algorithm 28 is configured to shape the outer surface of the bolus geometry to anonymize the sensitive anatomical region.

Input field 140 is for the user to enter a desired minimal buildup depth (referred to earlier as the goal buildup thickness), which is usually a function of the beam energy to be used. A higher beam energy generally requires a thicker buildup depth, whereas a lower beam energy requires a thinner buildup depth.

Input field 142 is for the user to enter the size of a desired external margin of tapered material to be added to the optimized thickness bolus 54. This external margin allows for connecting fixation devices, user labeling, and/or applying skin-safe adhesive to keep the optimized thickness bolus 54 in place on the patient.

Input field 144 is for the user to select the bolus material from which the bolus will be made. For example, the user may select a silicone-based material or a millable solid material or paraffin-based compound, such as blue wax.

Input field 146 is for the user to select alignment guides or no alignment guides. Alignment guides are on the outer (visible) surface of the optimized thickness bolus 54, and are used for visual alignment using treatment machine systems and/or room alignment systems, e.g., laser alignment systems. If the user needs alignment guides, the user is to select a type of alignment guides that are either concave (grooves) or convex (ridges).

If concave or convex alignment guides are selected, input field 148 is for the user to select the sagittal plane at which to add the alignment guides. The sagittal plane is an anatomical plane that divides the body into left and right portions, but in this context will provide alignment for viewers, lasers, or cameras from the anterior of the patient. This manifests as a guide running inferior (foot) to superior (head) and normal to the axial planes when viewed from normal incidence above (anterior) to the patient. Similarly, input field 150 is for the user to select coronal plane at which to add the alignment guides. The coronal plane is an anatomical plane that divides the body into front (anterior) and back (posterior) portions, and in this context will produce alignment guides for viewers, lasers, or cameras coming from the patient’s left and right.

Within the bolus design algorithm 28, Block 90 corresponds to the user identified primary purpose of the optimized thickness bolus 54. If the primary purpose is for target dose coverage and dosimetry, then the user-defined target regions of interest are not pre-processed, and the bolus design algorithm 28 proceeds to block 94.

However, if the primary purpose is for patient privacy and surface simplification, then the user-defined regions of interest are pre-processed at Block 92 to simplify the shape. This is done to remove details of the shape’s surface without altering its overall shape or volume. After the pre-processing, the bolus design algorithm 28 proceeds to block 94.

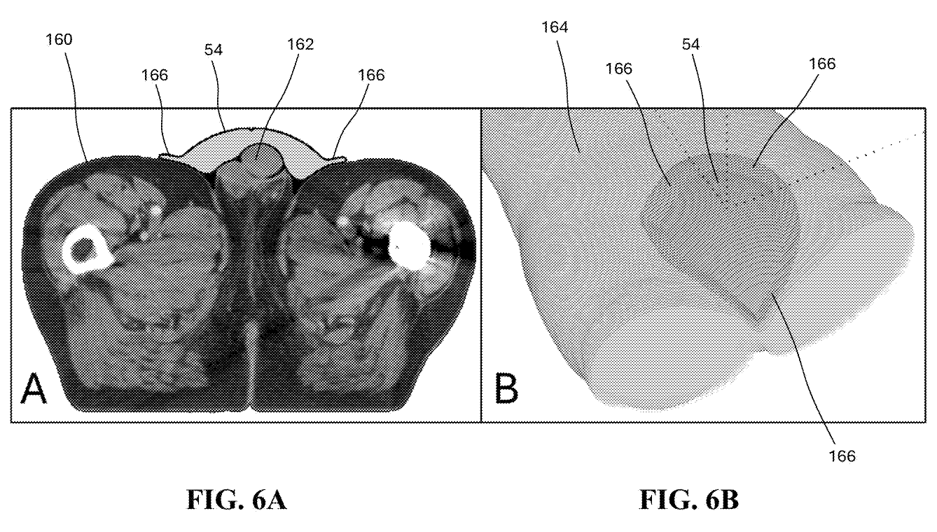

An axial CT view 160 of an optimized thickness bolus 54 used for the purpose of patient privacy and patient surface simplification (as seen to external photons beams) is illustrated in FIG. 6A. The CT image 40 is of a patient’s anterior pelvis which exposes male genitalia 162. The bolus design algorithm 28 is configured to shape the outer surface of the optimized thickness bolus 54 to anonymize the male genitalia 162.

A 3D view of the optimized thickness bolus 54 is illustrated in FIG. 6B protecting exposure of the male genitalia 162 of the patient 164. The outer surface of the optimized thickness bolus 54 has been smoothed so that it does not mimic the inner patient-facing surface of the patient. As such, the visible outer surface will hide sensitive anatomy rather than duplicating it. Patient privacy is ensured, and patients will feel less exposed and less self-conscious.

At Block 94, the axial contours of the target regions of interest are rendered into a 3D tagged orthogonal grid with sufficiently small point spacings (i.e., voxel sizes) to accurately capture even complex shapes. A voxel has a specific position in 3D space with a volume.

A voxel has one of three tag values, where the tag value will capture if each voxel is (a) inside, (b) on the surface, or (c) outside the target region of interest. The bolus design algorithm 28 models the target volume as a 3D grid rather than a series of axial contours. The contoured target volume is converted into a fine 3D grid of tiny, abutting rectangular boxes (voxels) so that its shape is captured accurately in all directions.

At Block 96, a one-dimensional (1D) target kernel is generated based on user inputs (e.g., desired goal buildup distance). In this context, a kernel is a 1D mathematical function or weighting pattern that is applied to input 3D grid data. The grid data in this case is the 3D voxel grid of the internal target volume. The kernel is used to expand and perturb, in 3D, the target grid at Block 98. This creates a heterogeneous grid of numeric values, which is then normalized based on target size/volume and buildup kernel. This is the initial bolus design grid.

Optionally, a superposition calculation can vary the 1D kernel across all voxels, and in all 3D vector directions from each voxel. This may be based on tissue density heterogeneities modeled from the image pixels, e.g., the CT values converted to dosimetrically important relative electron density. Superposition could be used in cases where the target has, or is surrounded by, very heterogeneous patient densities, e.g., containing low-density portions of lung or air or high-density portions like bone.

Similar to Step B for the target, the axial contours of the external region of interest are rendered into a 3D tagged orthogonal grid at Block 100 with the same dimensions and resolution of the initial bolus design grid. All voxels in the bolus design grid that are also “in” or “on” the patient external region of interest will be cropped from the bolus design grid at Block 102. This is effectively carving out the volume occupied by the patient body (and all internal organs and targets) from the bolus design. This is because the optimized thickness bolus 54 is purely an external structure abutting the patient, not inserted into the patient.

As a follow-up to Steps D1-D2, the bolus geometry includes an inner surface conforming to the external patient contour, and an outer surface determined based on the internal target volume and megavoltage photon beam dose deposition characteristics to be applied to the internal target volume. Corresponding to Block 100 (Step D1), an external shape is created to influence the megavoltage photon beam dose deposition characteristics in the target volume within the created external shape. Corresponding to Block 102 (Step D2), the external patient contour is subtracted from the created external shape at Block 102. This defines the bolus geometry for the optimized thickness bolus 54. As a result, a profile of the optimized thickness bolus 54 defines a spatially-varying thickness between the inner surface and the outer surface.

The patient external tagged grid (from Step D) is also used with a separate expansion kernel at Block 104 to expand a volume of nominal thickness outside the patient surface at Block 106. This expanded grid is cropped at the patient surface, similar to step D of the bolus design grid, to create a uniform shell of specified minimum thickness outside the patient.

This grid is unioned with the target-based bolus design grid (keeping largest value per-voxel, not summing them), then truncated based on the user-defined outer margin distance to be modeled at the edges of the bolus. The result at this point is a modified bolus design grid that has an outer margin of the specified minimum thickness outside the variable-thickness target-based design grid.

Referring to FIGS. 6A-6B, the outer edges of the optimized thickness bolus 54 will have a user-defined margin 166 of a specified minimum thickness outside of the bolus material that is needed dosimetrically. This margin allows for connecting fixation devices, user labeling, and/or applying skin-safe adhesive to the patients-side to keep the bolus in place, even if inverted with respect to the patient surface. The outer edges of the user-defined margin are tapered and smooth.

At Block 108, axial contours are extracted from the heterogeneous bolus design grid as if from a 3D topographical map, per axial slice. The extraction rules are part of the bolus design parameters and are internal (not user-defined) based on physics modeling. This step renders the bolus design from a finely-spaced 3D grid into a series of closed-loop contours on the original axial slice positions. That is, the bolus design is in the same form as the original input anatomical data.

At Block 110, the contours of the optimized thickness bolus 54 are post-processed to smooth them to better model the patient surface (remove artifacts if the external contours were digitized or blocky) and to taper the outer bolus edges to ensure there are no square or steep edges.

Also, any very small contours (also possible as artifacts of the input data) are removed from the optimized thickness bolus 54 at Block 112, as each bolus must be a contiguous volume. In other words, the bolus design algorithm 28 is configured to shape the inner surface of the bolus geometry to correct small-scale imperfections in the external patient contour, such that the photon beam bolus more accurately conforms thereto.

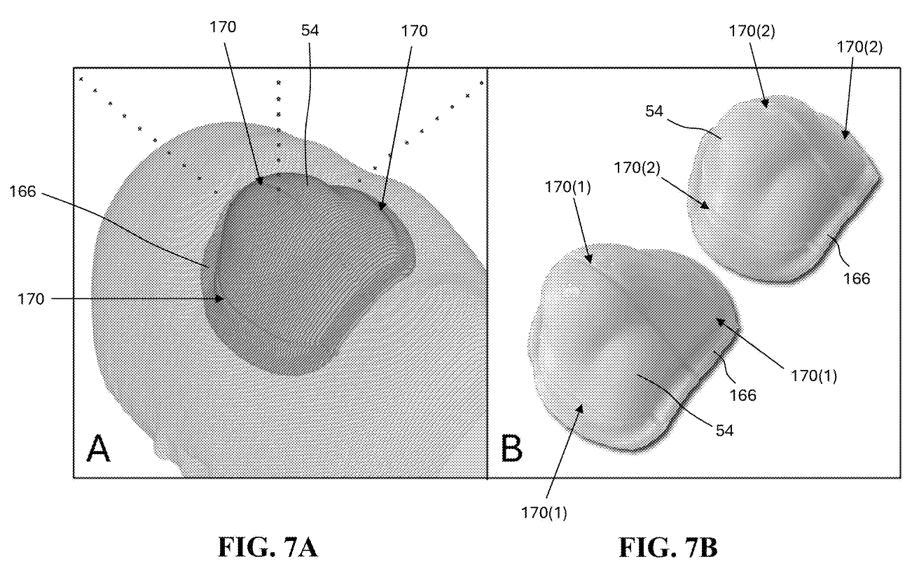

If the user-entered design parameters included alignment guides, then the alignment guides 170 are added to the optimized thickness bolus 54 at Block 114, as illustrated in FIGS. 7A-7B. The illustrated optimized thickness bolus 54 is configured to be placed on a patient’s head 171. The alignment guides are on the outer (visible) surface of the optimized thickness bolus 54, and are used for visual alignment using treatment machine systems and/or room alignment systems, e.g., laser alignment systems.

The alignment guides 170 may be configured to have a concave (grooves) shape 170(1) or a convex (ridges) shape 170(2), both of which are illustrated in FIG. 7B.

For the concave (grooves) shape 170(1), at the user-defined localization planes (e.g., sagittal or coronal planes), small circular region of interests are carved out of each axial contour to form the concave surface indentations that form the grooves in a perfect sagittal plane (if viewed from above or below) or coronal plane (if viewed laterally from left or right). Similarly, for the convex (ridges) shape 170(2), at the user-defined localization planes, small circular region of interests are added to each axial contour to form the convex surface indentations that form the grooves in a perfect sagittal plane (if viewed from above or below) or coronal plane (if viewed laterally from left or right).

Also illustrated in FIGS. 7A-7B, the outer edges of the optimized thickness bolus 54 have a user-defined margin 166 of a specified minimum thickness outside of the bolus material that is needed dosimetrically. In this case, the margin is a 10 mm conformal outer margin. As noted above, this margin allows for connecting fixation devices, user labeling, and/or applying skin-safe adhesive to the patients-side to keep the bolus in place. Moreover, the outer edges of the user-defined margin 166 are tapered and smooth.

Finally, the resulting axial contours of the bolus region of interest are analyzed with a separate algorithm at Block 116 to identify if there are any noncontiguous volumes. If there are noncontiguous volumes, each volume is separated into a separate bolus ROI. The bolus ROIs are saved as separate structures in the output DICOM RT Structure Set.

As a follow-up to Steps I, in response to the internal target volume corresponding to noncontiguous anatomical regions, the bolus design algorithm 28 is configured to generate a plurality of separate bolus geometries. Each bolus geometry corresponds to a respective region and is configured for independent manufacture. A user is enabled to select one or more of the plurality of bolus geometries for use. The DICOM RT Structure Set with the optimized thickness bolus or boluses 54 are available at Block 118.

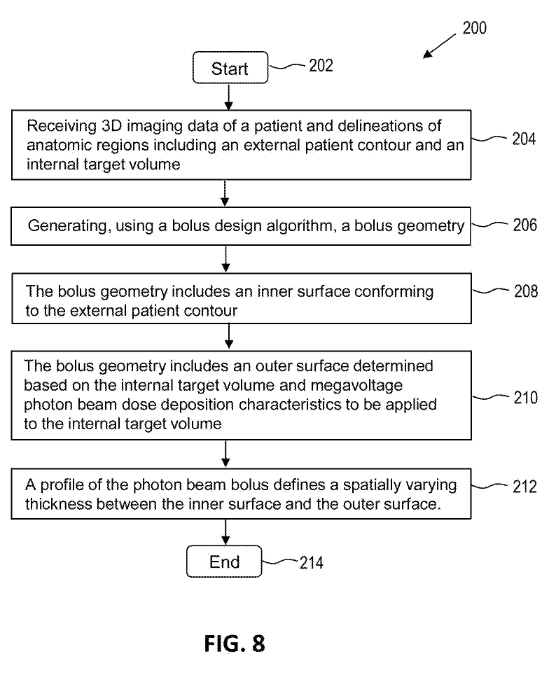

Another aspect is directed to a method for designing a photon beam bolus 54 as described above for use in photon beam radiation therapy. Referring now to the flowchart 200 in FIG. 8, from the start (Block 202), the method includes receiving three-dimensional imaging data of a patient and delineations of anatomic regions at Block 204 that includes an external patient contour and an internal target volume.

A bolus design algorithm 28 is used at Block 206 to generate a bolus geometry for the photon beam bolus 54. The bolus geometry includes an inner surface at Block 208 conforming to the external patient contour, and an outer surface determined at Block 210 based on the internal target volume and megavoltage photon beam dose deposition characteristics to be applied to the internal target volume. This results in a profile of the photon beam bolus being defined at Block 212 as a spatially-varying thickness between the inner surface and the outer surface. The method ends at Block 214.

Yet another aspect is directed to a non-transitory computer readable medium having computer-executable instructions for causing a computing device 20 to execute a bolus design algorithm 28.

Many modifications and other embodiments will come to the mind of one skilled in the art having the benefit of the teachings presented in the foregoing descriptions and the associated drawings. Therefore, it is understood that the foregoing is not to be limited to the example embodiments, and that modifications and other embodiments are intended to be included within the scope of the appended claims.

1. A method for designing a photon beam bolus for use in photon beam radiation therapy, the method comprising:
receiving three-dimensional imaging data of a patient and delineations of anatomic regions including an external patient contour and an internal target volume; and
generating, using a bolus design algorithm, a bolus geometry comprising:
an inner surface conforming to the external patient contour, and
an outer surface determined based on the internal target volume and megavoltage photon beam dose deposition characteristics to be applied to the internal target volume,
with a profile of the photon beam bolus defining a spatially-varying thickness between the inner surface and the outer surface.

2. The method according to claim 1 wherein the bolus design algorithm is configured to perform the following to generate the bolus geometry:
create an external shape to influence the megavoltage photon beam dose deposition characteristics in the target volume within the created external shape; and
subtract the external patient contour from the created external shape.

3. The method according to claim 1 wherein the bolus geometry is configured for any photon beam direction.

4. The method according to claim 1 wherein the bolus design algorithm is configured to add alignment guides to the outer surface of the photon beam bolus, with the alignment guides being used to accurately position the photon beam bolus on the patient.

5. The method according to claim 4 wherein the alignment guides are configured as grooves or ridges.

6. The method according to claim 1 wherein the external patient contour includes a sensitive anatomical region, and wherein the bolus design algorithm is configured to shape the outer surface of the bolus geometry to anonymize the sensitive anatomical region.

7. The method according to claim 1 wherein the bolus design algorithm is configured to apply a user-defined margin to the bolus geometry.

8. The method according to claim 7 wherein outer edges of the user-defined margin are tapered and smooth.

9. The method according to claim 1 wherein the bolus design algorithm is configured to shape the inner surface of the bolus geometry to correct small-scale imperfections in the external patient contour, such that the photon beam bolus more accurately conforms thereto.

10. The method according to claim 1 wherein, in response to the internal target volume corresponding to noncontiguous anatomical regions, the bolus design algorithm is configured to generate a plurality of separate bolus geometries, with each bolus geometry corresponding to a respective region and configured for independent manufacture, and wherein a user is enabled to select one or more of the plurality of bolus geometries for use.

11. The method according to claim 1 comprising exporting the bolus geometry for manufacture of the photon beam bolus.

12. The method according to claim 1 comprising exporting the bolus geometry to a treatment planning system.

13. A computing device comprising:
a memory configured to receive three-dimensional imaging data of a patient and delineations of anatomic regions including an external patient contour and an internal target volume; and
a processor coupled to the memory and configured to execute a bolus design algorithm for designing a photon beam bolus for use in photon beam radiation therapy, with the bolus design algorithm configured to generate a bolus geometry comprising:
an inner surface conforming to the external patient contour, and
an outer surface determined based on the internal target volume and megavoltage photon beam dose deposition characteristics to be applied to the internal target volume,
with a profile of the photon beam bolus defining a spatially-varying thickness between the inner surface and the outer surface.

14. The computing device according to claim 13 wherein the bolus design algorithm is configured to perform the following to generate the bolus geometry:
create an external shape to influence the megavoltage photon beam dose deposition characteristics in the target volume within the created external shape; and
subtract the external patient contour from the created external shape.

15. The computing device according to claim 13 wherein the bolus geometry is configured for any photon beam direction.

16. The computing device according to claim 13 wherein the bolus design algorithm is configured to add alignment guides to the outer surface of the photon beam bolus, with the alignment guides being used to accurately position the photon beam bolus on the patient.

17. The computing device according to claim 13 wherein the external patient contour includes a sensitive anatomical region, and wherein the bolus design algorithm is configured to shape the outer surface of the bolus geometry to anonymize the sensitive anatomical region.

18. The computing device according to claim 13 wherein the bolus design algorithm is configured to apply a user-defined margin to the bolus geometry.

19. The computing device according to claim 13 wherein the bolus design algorithm is configured to shape the inner surface of the bolus geometry to correct small-scale imperfections in the external patient contour, such that the photon beam bolus more accurately conforms thereto.

20. The computing device according to claim 13 wherein, in response to the internal target volume corresponding to noncontiguous anatomical regions, the bolus design algorithm is configured to generate a plurality of separate bolus geometries, with each bolus geometry corresponding to a respective region and configured for independent manufacture, and wherein a user is enabled to select one or more of the plurality of bolus geometries for use.

21. A photon beam bolus for use in photon beam radiation therapy, comprising:
a body comprising a bolus geometry including:
an inner surface configured to conform to an external patient contour; and
an outer surface defined based on an internal target volume and megavoltage photon beam dose deposition characteristics associated with the internal target volume;
wherein the bolus geometry defines a spatially-varying thickness between the inner surface and the outer surface.

22. A non-transitory computer readable medium having computer-executable instructions for causing a computing device to execute a bolus design algorithm, with the computing system to perform steps comprising:
receiving three-dimensional imaging data of a patient and delineations of anatomic regions including an external patient contour and an internal target volume; and
designing a photon beam bolus for use in photon beam radiation therapy, with the bolus design algorithm configured to generate a bolus geometry comprising:
an inner surface conforming to the external patient contour, and
an outer surface determined based on the internal target volume and megavoltage photon beam dose deposition characteristics to be applied to the internal target volume,
with a profile of the photon beam bolus defining a spatially-varying thickness between the inner surface and the outer surface.
